(19) United States
(12) Patent Application Publication
(10) Pub. No.: US 2013/0129787 A1
(43) Pub. Date: May 23, 2013

(54) ADHESIVE COMPLEX COACERVATES PRODUCED FROM ELECTROSTATICALLY ASSOCIATED BLOCK COPOLYMERS AND METHODS FOR MAKING AND USING THE SAME

(71) Applicant: Russell J. Stewart, Salt Lake City, UT (US)

(72) Inventor: Russell J. Stewart, Salt Lake City, UT (US)

(21) Appl. No.: 13/580,794

(22) PCT Filed: Feb. 25, 2011
(86) PCT No.: PCT/US11/26169

§ 371 Date: Feb. 7, 2013

Related U.S. Application Data
(60) Provisional application No. 61/308,454, filed on Feb. 26, 2010.

(51) Int. Cl. C07K 14/00

(52) U.S. Cl. CPC C07K 14/001
USPC 424/400; 530/353; 424/682; 424/641; 424/639; 424/653; 424/647; 514/16.7; 514/773; 514/20.8; 514/13.5; 106/124.1; 428/221

(57) ABSTRACT

Described herein is the synthesis of adhesive complex coacervates from electrostatically associated block copolymers, wherein the block copolymers comprise alternating polycationic and polyanionic blocks. Methods for making and the using the adhesive complex coacervates are also described herein.

### CROSS REFERENCE TO RELATED APPLICATION

This application claims priority upon U.S. provisional application Ser. No. 61/308,454, filed Feb. 26, 2010. This application is hereby incorporated by reference in its entirety for all of its teachings.

### ACKNOWLEDGEMENTS

The research leading to this invention was funded in part by the National Science Foundation Division of Materials Research, Grant No. 0906014. The U.S. Government has certain rights in this invention.

### BACKGROUND

Commercially important silkworm silk is comparatively well studied. The core of the fibers is comprised of heavy chain fibroin (H-fibroin, 250-500 kDa), light chain fibroin (L-fibroin, ˜25 kDa), and the glycoprotein P25 (˜30 kDa). These proteins are produced in posterior silk gland cells, assembled into elementary secretory units in a 6:6:1 molar ratio, and released from secretory granules into the silk gland lumen. The heavy and light chain fibroins are covalently linked through a single intermolecular disulfide bond. On the way to being drawn-out of labial spinnerets as an insoluble filament the concentrated fibroin suspension is coated with a heterogeneous mixture of sticky sericins, aligned into microfibrils, and possibly dehydrated as it passes through the middle and anterior regions of the silk gland. The final spun-out silk consists of two filaments from the paired silk glands fused into a single fiber coated with adhesive sericins.

Although silk produced from terrestrial insects like moths and silkworms has been studied extensively, far less is known about the silk produced by caddisflies. Caddisflies (order Trichoptera) are a large group of aquatic insects. They occupy freshwater habitats ranging from cold fast moving mountain streams to still marshes, often with several species dividing resources within each habitat. The larval stages feed, mature, and pupate underwater. The pupae “hatch” into short-lived winged adults that leave the water to mate. The caddisflies' successful penetration into diverse aquatic habitats is largely due to the use by their larva of underwater silk to build elaborate structures for protection and food gathering.

It would be desirable to produce synthetic analogues of the fibers produced by the caddisfly, as these fibers would have numerous applications as bioadhesives and in industrial applications.

### SUMMARY

Described herein is the synthesis of adhesive complex coacervates from electrostatically associated block copolymers, wherein the block copolymers comprise alternating polycationic and polyanionic blocks. Methods for making and using the adhesive complex coacervates are also described herein. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 shows the electrostatic interaction between electrostatically associated block copolymers to produce a synthetic fiber herein.

FIG. 2 shows: (A) Brachycentrus echo larva in a case partially constructed with glass beads in a laboratory aquarium. (B-D) SEMs of the inside of the glass case at increasing magnification (Scale bars: 500, 250, 100 microns, respectively). (E) SEM of the region in F analyzed by EDS. Blue=phosphorus, purple=silicon (scale bars: 10 microns).

FIG. 3 shows: (A) Western blot of silk proteins with anti-pS antibody. Lane 1: caddisfly (B. echo) silk extracted from dissected silk glands with 8M urea, Lane 2: caddisfly silk extracted with SDS, Lane 3: silkworm (B. mori) silk extracted from dissected silk glands with 8M urea, Lane 4: silkworm silk extracted with SDS. (B) B. echo larval silk gland immunostain control. (C) Larval silk glands immunostained with anti-pS antibody. The head (dark object) is still attached to the paired silk glands. Staining occured only in the posterior region of the intact silk glands. (D) Anti-pS control. B. echo silk fibers were treated as in E without the anti-pS primary antibody. (E) B. echo silk fibers on glass beads labelled with anti-pS antibody.

FIG. 4 shows a schematic diagram of hypothetical repeating domain structure formed by phosphoserine and Ca²⁺.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Examples of longer chain alkyl groups include, but are not limited to, a palmitate group. A “lower alkyl” group is an alkyl group containing from one to six carbon atoms.

Any of the block copolymers useful herein can be the pharmaceutically-acceptable salt. In one aspect, pharmaceutically-acceptable salts are prepared by treating the free acid with an appropriate amount of a pharmaceutically-acceptable base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

In another aspect, if the block copolymer possesses a basic group, it can be protonated with an acid such as, for example, HCI, HBr, or H₂SO₄, to produce the cationic salt. In one aspect, the reaction of the block copolymer with the acid or base is conducted in water, alone or in combination with an inert, water-miscible organic solvent, at a temperature of from about 0° C. to about 100° C. such as at room temperature. In certain aspects where applicable, the molar ratio of the block copolymer described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

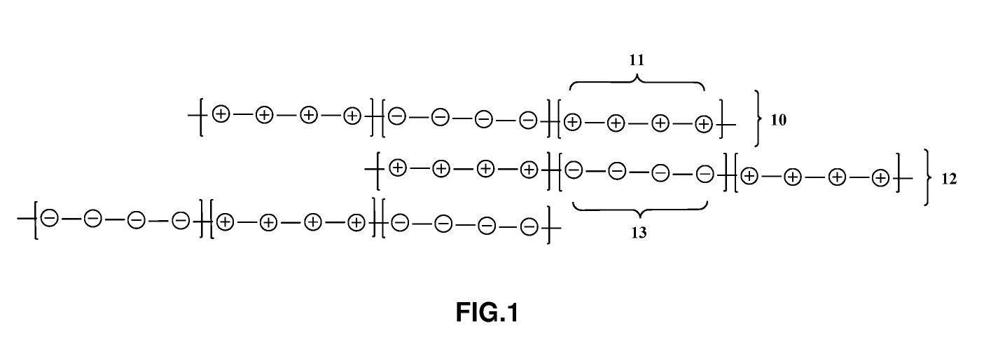

Described herein are adhesive complex coacervates produced from electrostatically associated block copolymers and their applications thereof. The electrostatically associated block copolymers are water-soluble polymers composed of alternating polycationic blocks and polyanionic blocks. The polycationic blocks of one copolymer are electrostatically attracted to one or more polyanionic blocks present in another block copolymer. This is depicted in FIG. 1, where the polycationic block 11 of copolymer 10 is electrostatically attracted to the polyanionic block 13 in copolymer 12. The examples provide a detailed analysis of the fibers produced by the caddisfly, which exhibited similar patterns of positively and negatively charged blocks of groups. As discussed in detail below, when the net charge of the copolymers approaches neutral, the block copolymers form an insoluble material in water. This feature of the adhesive complex coacervates described herein have numerous applications as an adhesive, particularly a medical adhesive.

The adhesive complex coacervate is an associative liquid with a dynamic structure in which the individual copolymer components diffuse throughout the entire phase. Complex coacervates behave rheologically like viscous particle dispersions rather than a viscoelastic polymer solution. As described above, the adhesive complex coacervates exhibit low interfacial tension in water when applied to substrates either under water or that are wet. In other words, the complex coacervate spreads evenly over the interface rather than beading up.

The block copolymers are generally composed of a polymer backbone with alternating polycationic blocks (i.e., blocks having a net positive charge) and polyanionic blocks (i.e., blocks having a net negative charge). Individual positive or negative charged groups are present in each block. The groups can be pendant to the polymer backbone and/or incorporated within the polymer backbone. In certain aspects, (e.g., biomedical applications), the polycationic blocks are composed of a series of cationic groups or groups that can be readily converted to cationic groups by adjusting the pH. In one aspect, the polycationic block is a polyamine compound. The amino groups of the polyamine can be branched or part of the polymer backbone. The amino group can be a primary, secondary, tertiary, or a guanidinium group that can be protonated to produce a cationic ammonium group at a selected pH.

In one aspect, the polycationic block of the copolymer can be derived from residues of lysine, histidine, arginine, and/or imidazole. Any anionic counterions can be used in association with the polycationic block. The counterions should be physically and chemically compatible with the essential components of the composition and do not otherwise unduly impair product performance, stability or aesthetics. Non-limiting examples of such counterions include halides (e.g., chloride, fluoride, bromide, iodide), sulfate and methylsulfate.

In another aspect, the polycationic block can be a biodegradable polyamine. The biodegradable polyamine can be a synthetic polymer or naturally-occurring polymer. The mechanism by which the polyamine can degrade will vary depending upon the polyamine that is used. In the case of natural polymers, they are biodegradable because there are enzymes that can hydrolyze the polymers and break the polymer chain. For example, proteases can hydrolyze natural proteins like gelatin. In the case of synthetic biodegradable polyamines, they also possess chemically labile bonds. For example, β-aminoesters have hydrolyzable ester groups. In addition to the nature of the polyamine, other considerations such as the molecular weight of the polyamine and crosslink density of the adhesive can be varied in order to modify the degree of biodegradability.

In one aspect, the biodegradable polyamine includes a polysaccharide, a protein, a peptide, or a synthetic polyamine. Polysaccharides bearing one or more amino groups can be used herein. In one aspect, the polysaccharide is a natural polysaccharide such as chitosan. Similarly, the protein can be a synthetic or naturally-occurring compound. In another aspect, the biodegradable polyamine is a synthetic polyamine such as poly(β-aminoesters), polyester amines, poly(disulfide amines), mixed poly(ester and amide amines), and peptide crosslinked polyamines.

In the case when the polycationic block is a synthetic polymer, a variety of different polymers can be used; however, in certain applications such as, for example, biomedical applications, it is desirable that the polymer be biocompatible and non-toxic to cells and tissue. In one aspect, the biodegradable polyamine can be an amine-modified natural polymer. For example, the amine-modified natural polymer can be gelatin modified with one or more alkylamino groups, heteroaryl groups, or an aromatic group substituted with one or more amino groups. Examples of alkylamino groups are depicted in Formulae III-V

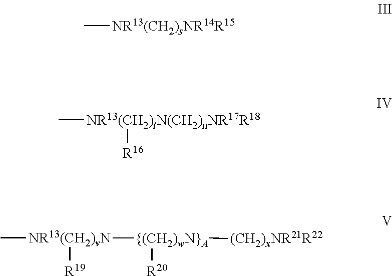

wherein R¹³—R²² are, independently, hydrogen, an alkyl group, or a nitrogen containing substituent;
s, t, u, v, w, and x are an integer from 1 to 10; and
A is an integer from 1 to 50,
where the alkylamino group is covalently attached to the natural polymer. In one aspect, if the natural polymer has a carboxyl group (e.g., acid or ester), the carboxyl group can be reacted with a polyamine compound to produce an amide bond and incorporate the alkylamino group into the polymer. Thus, referring to formulae III-V, the amino group NR¹³ is covalently attached to the carbonyl group of the natural polymer.

As shown in formula III-V, the number of amino groups can vary. In one aspect, the alkylamino group is —NHCH₂NH₂, —NHCH₂CH₂NH₂, —NHCH₂CH₂CH₂NH₂, —NHCH₂CH₂CH₂CH₂NH₂, —NHCH₂CH₂CH₂CH₂CH₂NH₂, —NHCH₂NHCH₂CH₂CH₂NH₂, —NHCH₂CH₂NHCH₂CH₂CH₂NH₂, —NHCH₂CH₂CH₂NHCH₂CH₂CH₂CH₂NHCH₂CH₂CH₂NH₂, —NHCH₂CH₂NHCH₂CH₂CH₂CH₂NH₂, —NHCH₂CH₂NHCH₂CH₂CH₂NHCH₂CH₂CH₂NH₂, or —NHCH₂CH₂NH(CH₂CH₂NH)_{d}CH₂CH₂NH₂, where d is from 0 to 50.

In one aspect, when the polycationic block is an amine-modified natural polymer, the amine-modified natural polymer can include an aryl group having one or more amino groups directly or indirectly attached to the aromatic group. Alternatively, the amino group can be incorporated in the aromatic ring. For example, the aromatic amino group is a pyrrole, an isopyrrole, a pyrazole, imidazole, a triazole, or an indole. In another aspect, the aromatic amino group includes the isoimidazole group present in histidine. In another aspect, the biodegradable polyamine can be gelatin modified with ethylenediamine.

In one aspect, the polycationic block includes a polyacrylate having one or more pendant amino groups. For example, the backbone of the polycationic block can be a homopolymer or copolymer derived from the polymerization of acrylate or methacrylate monomers.

In other aspects, the polycationic block can in itself be a copolymer (i.e., random or block), where segments or portions of the copolymer possess cationic groups depending upon the selection of the monomers used to produce the copolymer. In this aspect, the number of positively charged groups present in the polycationic block can vary from a few percent up to 100 percent (e.g., between 10 and 50%). In this aspect, the polycationic block can be the polymerization product between a neutral monomer (i.e., no charged groups) and a monomer possessing a positively charged group, where the amount of each monomer will determine the overall positive charge of the polycationic block. Thus, it is possible to produce different polycationic blocks within the electrostatically associated block copolymer. Equations 1-3 below depict different embodiments regarding the polyactionic block. In equation 1, the same polycationic block (A) is incorporated into the block copolymer. In equation 2, two different polycationic blocks (A and B) are present in each polycationic block. In the case of the polycationic block AB in equation 2, monomers possessing different cationic groups can be used to produce the polycationic block AB. Thus, the polycationic block can in itself be a block copolymer. This is depicted in equation 2, where A depicts the first block in the polyactionic block and B depicts the second block. In equation 3, there are two different polycationic blocks, where each block (A and B) is the polymerization product of the same monomer.

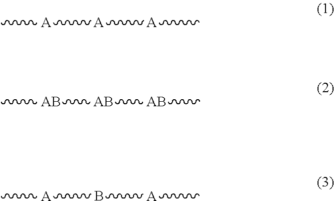

In one aspect, the polycationic block has at least one fragment of the formula I

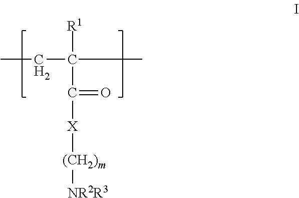

wherein R¹, R², and R³ are, independently, hydrogen, an alkyl group, or a guanidinium group [—C═NH(NH₂)], X is oxygen or NR⁵, where R⁵ is hydrogen or an alkyl group, and m is from 1 to 10, or the pharmaceutically-acceptable salt thereof. In another aspect, R¹, R², and R³ are methyl and m is 2. In another aspect R² is hydrogen and R³ is a guanidinium group. Referring to formula I, the polymer backbone of the polycationic block is composed of CH₂—CR¹ units with pendant —C(O)X(CH₂)ₘNR²R³ units. In this aspect, the fragment having the formula I is a residue of an acrylate or methacrylate.

Similar to the polycationic block, the polyanionic block in the copolymers described herein can be a synthetic polymer. The polyanionic block is generally any polymer possessing anionic groups or groups that can be readily converted to anionic groups by adjusting the pH. Examples of groups that can be converted to anionic groups include, but are not limited to, carboxylate, sulfonate, phosphonate, boronate, sulfate, borate, or phosphate. Any cationic counterions can be used in association with the anionic polymers if the considerations discussed above are met.

The polycationic block can in itself be a copolymer (i.e., random or block), where segments or portions of the copolymer possess cationic groups depending upon the selection of the monomers used to produce the copolymer. In this aspect, the number of negatively charged groups present in the polyanionic block can vary from a few percent up to 100 percent (e.g., between 10 and 50%). In this aspect, the polyanionic block can be the polymerization product between a neutral monomer (i.e., no charged groups) and a monomer possessing a negatively charged group, where the amount of each monomer will determine the overall negative charge of the polyanionic block. Thus, it is possible to produce different polyanionic blocks within the electrostatically associated block copolymer.

In one aspect, the polyanionic block is a polyphosphate. In another aspect, the polyanion is a polyphosphate compound having from 10 to 90 mole % phosphate groups (i.e., a random copolymer). For example, the polyphosphate can be a polymer with pendant phosphate groups attached to the polymer backbone of the polyanionic block and/or present in the polymer backbone of the polyanionic block (e.g., a phosphodiester backbone). In one aspect, the polyphosphate can be produced by chemically or enzymatically phosphorylating a protein (e.g., natural serine-rich proteins).

In one aspect, the polyanionic block includes a polyacrylate having one or more pendant phosphate groups. For example, the backbone of the polyanionic block can be a homopolymer or copolymer derived from the polymerization of acrylate monomers including, but not limited to, acrylates and methacrylates, Similar to above for the polycationic blocks as shown in equations 1-3, the polycationic blocks can be composed of the same or different blocks (A and B).

In one aspect, the polyanionic block is a polyphosphate. In another aspect, the polyanionic block is a polymer having at least one fragment having the formula II

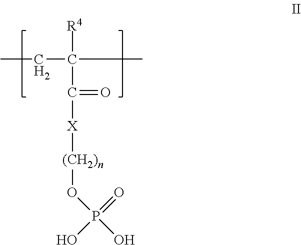

wherein R⁴ is hydrogen or an alkyl group, X is oxygen or NR⁵, where R⁵ is hydrogen or an alkyl group, and n is from 1 to 10, or the pharmaceutically-acceptable salt thereof. In another aspect, wherein R⁴ is methyl and n can be 2, 3, or 4. Similar to formula VII, the polymer backbone of formula II is composed of a residue of an acrylate or methacrylate. The remaining portion of formula II is the pendant phosphate group.

In certain aspects, the polycationic and polyanionic blocks contain groups that permit crosslinking between the different copolymers upon curing to produce new covalent bonds and the synthetic fiber. The mechanism of crosslinking can vary depending upon the selection of the crosslinking groups. In one aspect, the crosslinking groups can be electrophiles and nucleophiles. For example, the polyanionic block can have one or more electrohilic groups, and the polycationic block can have one or more nucleophilic groups capable of reacting with the electrophilic groups to produce new covalent bonds. Examples of electrophilic groups include, but are not limited to, anhydride groups, esters, ketones, lactams (e.g., maleimides and succinimides), lactones, epoxide groups, isocyanate groups, and aldehydes. Examples of nucleophilic groups are presented below.

In another aspect, the polycationic and polyanionic blocks each have an actinically crosslinkable group. As used herein, “actinically crosslinkable group” in reference to curing or polymerizing means that the crosslinking between the polycation and polyanion is performed by actinic irradiation, such as, for example, UV irradiation, visible light irradiation, ionized radiation (e.g. gamma ray or X-ray irradiation), microwave irradiation, and the like. Actinic curing methods are well-known to a person skilled in the art. The actinically crosslinkable group can be an unsaturated organic group such as, for example, an olefinic group. Examples of olefinic groups useful herein include, but are not limited to, an acrylate group, a methacrylate group, an acrylamide group, a methacrylamide group, an allyl group, a vinyl group, a vinylester group, or a styrenyl group.

In other aspects, the crosslinkers present on the polycationic and/or polyanionic blocks can form coordination complexes with transition metal ions. For example, a transition metal ion can be added to the copolymer, where the copolymer contains crosslinkers capable of coordinating with the transition metal ion. The rate of coordination and dissociation can be controlled by the selection of the crosslinker, the transition metal ion, and the pH. Transition metal ions such as, for example, iron, copper, vanadium, zinc, and nickel can be used herein.

In one aspect, the polycationic block can be a polyacrylate having one or more pendant amino groups (e.g., imidazole groups). In the case of the polyanionic block, in one aspect, a polyphosphate can be modified to include the actinically crosslinkable group(s). A spectrum of covalent crosslinking can be achieved using activated esters, including N-hydroxysuccinimide ester, imidazolyl carbamate derivatives and others. In certain aspects, thiopyridine derivatives, maleimide, and others can be included as crosslinkable moieties onto a polyphosphate copolymer to originate an adhesive with suitable mechanical properties. For example, the polycationic block includes at least one fragment having the formula I discussed above, wherein at least one of R² or R³ is an actinically crosslinkable group.

In certain aspects, the block copolymers composed of alternating polycationic blocks and polyanionic blocks can be crosslinked with one another to produce adhesive complex coacervates by controlling changes in temperature. In one aspect, the use of a thermoreversible Diels-Alder reaction can be used to crosslink the copolymers. In this aspect, ring coupling between a dienophile and a conjugated diene (e.g., furan and maleimide groups) can occur by increasing the temperature without the need of any chemical catalysts or initiators. Additionally, the presence of water can accelerate the reaction rate. The dienophile and a conjugated diene can be present on the polycationic blocks and/or polyanionic blocks.

In another aspect, the crosslinkable group includes a dihydroxyl-substituted aromatic group capable of undergoing oxidation in the presence of an oxidant. In one aspect, the dihydroxyl-substituted aromatic group is a dihydroxyphenol or halogenated dihydroxyphenol group such as, for example, DOPA and catechol (3,4 dihydroxyphenol). For example, in the case of DOPA, it can be oxidized to dopaquinone. Dopaquinone is an electrophilic group that is capable of either reacting with a neighboring DOPA group or another nucleophilic group. In the presence of an oxidant such as oxygen or other additives including, but not limited to, peroxides, periodates (e.g., NaIO₄), persulfates, permanganates, dichromates, transition metal oxidants (e.g., a Fe⁺³ compound, osmium tetroxide), or enzymes (e.g., catechol oxidase), the dihydroxyl-substituted aromatic group can be oxidized. In another aspect, crosslinking can occur between the polycation and polyanion via light activated crosslinking through azido groups. Once again, new covalent bonds are formed during this type of crosslinking.

In certain aspects, the oxidant can be stabilized. For example, a compound that forms a coordination complex with periodate that is not redox active can result in a stabilized oxidant. In other words, the periodate is stabilized in a non-oxidative form and cannot oxidize the dihydroxyl-substituted aromatic group while in the complex. The coordination complex is reversible and even if it has a very high stability constant there is a small amount of uncomplexed periodate present. The dihydroxyl-substituted aromatic group competes with the compound for the small amount of free periodate. As the free periodate is oxidized more is released from the reversible complex. In one aspect, sugars possessing a cis,cis-1,2,3-triol grouping on a six-membered ring can form competitive periodate complexes. An example of a specific compound that forms stable periodate complex is 1,2-O-isopropylidene-alpha-D-glucofuranose. The stabilized oxidant can control the rate of crosslinking. Not wishing to be bound by theory, the stabilized oxidant slows down the rate of oxidation so that there is time to add the oxidant and position the substrate before the fiber (i.e., adhesive) hardens irreversibly.

The stability of the oxidized crosslinker can vary. For example, the phosphono containing polyanionic blocks described herein can contain oxidizable crosslinkers that are stable in solution and do not crosslink with themselves. This permits nucleophilic groups present on the polycationic blocks to react with the oxidized crosslinker This is a desirable feature, which permits the formation of intermolecular bonds and, ultimately, the formation of a strong adhesive. Examples of nucleophilic groups that are useful include, but are not limited to, hydroxyl, thiol, and nitrogen containing groups such as substituted or unsubstituted amino groups and imidazole groups. For example, residues of lysine, histidine, and/or cysteine or chemical analogs can be incorporated into the polycationic block and introduce nucleophilic groups.

The coacervates can optionally contain one or more multivalent cations (i.e., cations having a charge of +2 or greater). In one aspect, the multivalent cation can be a divalent cation composed of one or more alkaline earth metals. For example, the divalent cation can be a mixture of Ca⁺² and Mg⁺². In other aspects, transition metal ions with a charge of +2 or greater can be used as the multivalent cation. In addition to the pH, the concentration of the multivalent cations can determine the rate and extent of fiber formation in water. The amount of multivalent cation used herein can vary. In one aspect, the amount is based upon the number of anionic groups and cationic groups present in the polyanionic blocks and polycationic blocks, respectively.

The copolymers described herein can be produced using techniques known in the art. For example, the reversible addition fragmentation chain transfer (RAFT) polymerization allows precise synthesis of block copolymers with acrylate and methacrylate monomers. In the RAFT method, primary radicals are generated as in conventional free radical polymerization with thermal, photochemical, or chemical redox initiators. RAFT polymerization is performed in the presence of a chain transfer agent (CTA) such as, for example, a dithioester of the form (S═C(Z)—S—R), which has higher reactivity than the monomer with free radicals. The CTA reversibly adds to the primary initiator radicals to create an intermediate radical species that fragments into a new CTA (macro-CTA) and a CTA derived radical (R.) that reinitiates polymerization. As the reaction progresses, a steady state is established in which the CTA is rapidly and reversibly transferred between dormant and propagating polymer chains, the effect of which is to prevent radical dimerization and disproportionation reactions that prematurely terminate polymer chains creating polymers with broad polydispersity. In successful living polymerizations, polymer chains are initiated rapidly then grow relatively slowly at a constant rate resulting in a linear increase in polymer mass and leading to polymers with narrow polydispersity.

In one aspect, copolymers with alternating polycationic and polyanionic blocks can be produced by RAFT polymerization by feeding a comonomer (e.g., an acrylate having a cationic group) into a polymerization reaction with a second comonomer (e.g., an acrylate having an anionic group) during the linear growth phase. Taking into account the relative reactivities, each comonomer can be fed at a programmed rate to alter the composition along the chain in a defined manner. A constant feed rate of one comonomer, for example, would result in a gradient copolymer. Thus, by altering the comonomer ratios at different times during chain elongation, the size and distribution of polycationic and polyanionic blocks in the copolymer can be manipulated.

In another aspect, the macro-CTA complex produced after the synthesis of a block is isolated then polymer propagation is reinitiated with a different monomer. For example, a phosphate block (polyanionic block Y) could be RAFT polymerized, isolated, and then extended with an amine-containing monomer (polycationic block Z) to create an YZ diblock copolymer. Thus, an YZ-copolymer could be created by repeating this process. To incorporate a protein, peptide, or other natural polymer as a block in a block copolymer, a RAFT agent can be synthesized on or conjugated to a protein, peptide, or natural polymer. The resulting construct can be used as a macro-CTA to initiation of polymerization of a charged block onto the protein or peptide or other natural polymer.

The adhesive complex coacervates can be produced by admixing one or more electrostatically associated block copolymers in water under controlled pH and temperature. At this point, the coacervate can be easily handled and administered as needed. By varying conditions such as, for example, pH and temperature, it is possible to convert the coacervate to a water insoluble material. For example, the coacervate can be extruded through a cannula into water at controlled temperature and pH using a syringe pump to produce fibers or filaments. In this aspect, the wetspinning of caddisfly silk analogs is simulated (see Examples), where the adhesive complex coacervate can form water-insoluble fibers. Not wishing to be bound by theory, staggered electrostatic association of alternating blocks with opposite charges present in the copolymers may drive liquid-liquid phase separation as complex coacervates. Complex coacervation and fiber formation occurs when oppositely charged polyelectrolytes associate in aqueous solution through mutual charge neutralization. When the solution is near net charge neutrality a dense concentrated polymer aqueous phase separates from a polymer depleted aqueous phase driven in part by entropic gains from the release of small counter ions and water. In subsequent steps during the fiber extrusion process, stress-induced elongation and reorganization of the coacervated copolymer phase could lead to nanofibril formation, additional charge neutralization and dehydration of the fiber during extrusion into water. The fibers could then be spun into two dimensional fabrics.

The properties of the adhesive complex coacervates described herein make them ideal adhesives in wet conditions. For example, the adhesive complex coacervates can be used as pressure sensitive adhesives. For example, the adhesive complex coacervate can be applied directly as a coating on the surface of a backing material (e.g., plastic), which can subsequently be adhered to a wet or moist substrate. Here, the adhesive complex coacervate behaves like a “wet band-aid.” Alternatively, the coacervate can be extruded as fibers on the backing as discussed to produce the pressure sensitive adhesive. Thus, in these aspects, the adhesive complex coacervates and fibers produced therefrom have numerous applications as medical adhesives.

In one aspect, the adhesive complex coacervates and fibers produced therefrom can be used to secure scaffolds to bone and other tissues such as, for example, cartilage, ligaments, tendons, soft tissues, organs, and synthetic derivatives of these materials. The adhesive complex coacervates and fibers can be used to position biological scaffolds in a subject. In certain aspects, the scaffold can contain one or more drugs that facilitate growth or repair of the bone and tissue. In other aspects, the scaffold can include drugs that prevent infection such as, for example, antibiotics. For example, the scaffold can be coated with the drug or, in the alternative, the drug can be incorporated within the scaffold so that the drug elutes from the scaffold over time.

In one aspect, the coacervate includes an astringent to reduce or stop bleeding at a surgical site. Thus, in addition to help seal one or more tissues cut during a surgical procedure, the coacervates can reduce or prevent hemostasis. Examples of astringents inorganic salts of aluminum, iron, zinc, manganese, bismuth, etc., as well as other salts containing these metals such as permanganates. Nonlimiting examples of suitable hemostatic astringents include ferric sulphate, ferric subsulphate, ferric chloride, zinc chloride, aluminum chloride, aluminum sulfate, aluminum chlorohydrate, and aluminum acetate. Alums such as aluminum potassium sulfate and aluminum ammonium sulfate may also be used. In addition, tannins or other related polyphenolic compounds may be used as the astringent. In certain aspects, the astringent can facilitate curing of the coacervate and stop bleeding. For example, ferric sulfate can perform this function.

In other aspects, the adhesive complex coacervates and fibers produced therefrom can adhere a metal substrate to bone. For example, implants made from titanium oxide, stainless steel, or other metals are commonly used to repair fractured bones. The adhesive complex coacervates and fibers produced therefrom can be applied to the metal substrate, the bone, or both prior to adhering the substrate to the bone. In certain aspects, a crosslinking group present on the polycationic or polyanionic block can form a strong bond with titanium oxide. For example, it has been shown that DOPA can strongly bind to wet titanium oxide surfaces (Lee et al., PNAS 103:12999 (2006)). Thus, in addition to bonding bone fragments, the adhesive complex coacervates and fibers produced therefrom can facilitate the bonding of metal substrates to bone, which can facilitate bone repair and recovery. In addition to metal substrates, the adhesive complex coacervates and fibers produced therefrom can be applied to other substrates such as, for example, backing materials, plastic films, or foils.

It is also contemplated that the adhesive complex coacervates and fibers produced therefrom can encapsulate one or more bioactive agents. The rate of release can be controlled by the selection of the materials used to prepare the complex as well as the charge of the bioactive agent if the agent is a salt.

For example, when the adhesive complex coacervates converted to a water insoluble material (i.e, synthetic fibers) by a change in temperature and/or pH, the adhesive complex coacervate can be administered to a subject and produce the insoluble material in situ. Thus, in this aspect, the water insoluble material can perform as a localized controlled drug release depot. It may be possible to simultaneously fix tissue and bones as well as deliver bioactive agents to provide greater patient comfort, accelerate bone healing, and/or prevent infections.

The adhesive complex coacervates and fibers can be used in a variety of other surgical procedures. For example, they can be used to repair lacerations caused by trauma or by the surgical procedure itself. In one aspect, the adhesive complex coacervates and fibers can be used to repair a corneal laceration in a subject. In other aspects, the adhesive complex coacervates and fibers can be used to inhibit blood flow in a blood vessel of a subject. In one aspect, the adhesive complex coacervate is injected into the vessel followed by conversion of the coacervate into a water insoluble material, which can partially or completely block the vessel. This method has numerous applications including hemostasis or the creation of an artificial embolism to inhibit blood flow to a tumor or aneurysm.

In addition to biomedical applications, the adhesive complex coacervates and fibers described herein have numerous applications in industrial applications. In general, the adhesive complex coacervates and fibers can be added to any composition that is applied to a substrate that is wet or moist. As discussed above, the adhesive complex coacervates and fibers enhance the adhesion of the composition to the wet or moist substrate. For example, the adhesive complex coacervates and fibers can be added to water-based compositions like paint. In this aspect, the adhesive complex coacervates and fibers enhance the bond between the paint and the substrate.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Sample preparation. Brachycentrus echo caddisfly larvae were collected from the lower Provo River, Utah, USA. Larvae with natural cases were maintained in an aquarium with circulating distilled water at 12° C. The natural stone cases were either partially or completely removed with fine forceps. The larva was then placed on a bed of pre-washed 0.5 mm glass beads in a clean glass vial. After 1-2 days glass cases either built onto the end of a natural case or completely rebuilt with glass beads were taken away from the larva that would start over on rebuilding their case. The harvested glass cases were frozen at −80° C., lyophilized, and then mounted on conductive carbon tape for SEM and EDS analysis (FEI Company, Quanta 600 FEG).

Amino acid and elemental analysis. Glass beads from reconstructed cases were carefully examined for contaminating sand or minerals. After lyophilization, weighed samples of beads with silk and without silk collected from the same vial were digested in 500 ml of 5.7 N HCl with 0.1% phenol in vacuo for 24 hrs at 110° C. An aliquot of the hydrolysate was analyzed for amino acids (Beckman 6300) and a second aliquot from the same hydrolysate was analyzed for metals by ICP-OES (PerkinElmer, Optima 3100XL) after dilution in 40% nitric acid. Elements were quantified by comparison to standard curves prepared with commercial mixed metal standards (PerkinElmer).

Gel electrophoresis and Western blot analysis. Dissected silk glands were transferred to a clean eppendorf tube containing DI water at 4° C. Silk proteins were released from the gland within 20 mins. To collect soluble fraction of silk proteins, samples were spun at 13,000 rpm for 5 mins at room temperature. The supernatant was transferred to a new tube for protein concentration determination (Bio-Rad). Forty mg of soluble silk proteins were subjected to SDS-PAGE on 15% gels. For Western blot analysis, the separated proteins were transferred to PVDF membranes that were then blocked with 2 mg/mL BSA in phosphate buffered saline (pH 7.4) at room temperature for at least 2 hrs. The blots were probed with an anti-phosphoserine mouse antibody (Abcam, #PSR45, 1:1000) at 4° C. overnight. After incubation with Horse radish peroxidase goat anti-mouse-HRP (1:5000) secondary antibody (Jackson Immuno Research, #115-035-166) at room temperature for 1 hr signals were developed with ECL (Pierce, #32109).

Silk gland immunostaining. Larvae were killed with 7% ethanol in DI water before the paired silk glands were removed still attached to the head. The glands were fixed with 4% paraformaldehyde in PBS at room temperature for 30 mins before permeabilization with proteinase digestion buffer (2 ug/mL proteinase K, 1% SDS, 0.1% Triton X-100 in PBS) at room temperature for 15 mins. The silk gland was blocked with 2 mg/mL BSA in PBS at room temperature for at least 2 hrs and then incubated with anti-pS antibody (Abcam, #PSR45, 1:1000) at room temperature for 1 hr. The primary antibody was labelled with a goat anti-mouse alkaline phosphatase conjugated secondary antibody (Abcam, #6729, 1:5000) at RT for another 1 hr. The blue signal was developed with NBT/BCIP (3:1 molar ratio) in AP buffer (150 mM NaCl, 100 mM Tris (pH 8.8), 5 mM MgCl₂ and 0.05% Tween-20) until blue color appeared. Glands were then dehydrated with serial dilution of ethanol (100%, 70%, 50% and TBS) to remove non-specific staining and followed by serial hydration for photo imagining.

Tandem mass spectrometry. Silk proteins were isolated from dissected B. echo silk glands in 25 mM ammonium bicarbonate. The silk proteins were heat denatured at 100° C., quickly cooled on ice to limit renaturation and digested with trypsin at an ˜1:25 ratio of enzyme to silk protein for 2 hrs at 37° C. Phosphopeptides from the silk protein digests were enriched by immobilized metal affinity chromatography (IMAC) using a SwellGel Gallium Disc (Pierce) according to the manufacturers instructions for phosphopeptide enrichment. The IMAC enriched peptides were analyzed by LC/MS/MS using a LTQ-FT hybrid mass spectrometer (ThermoElectron Corp). Peptides were introduced into the spectrometer by nanoLC (Eksigent, Inc.) using a C18 nanobore column and nano-electrospray ionization (ThermoElectron Corp). Peptides were eluted with a 50 min linear gradient of 5-60% acetonitrile with 0.1% formic acid. Primary peptide molecular masses were determined by FT-ICR and peptide sequences by collision-induced dissociation in the linear ion trap of the LTQ-FT hybrid mass spectrometer. Peptides were identified by MS/MS search using the Mascot search engine (ver. 2.2.1, Matrix Science). Possible phosphorylation on S, T, and Y were included in the search. Mascot thresholds were primary mass errors of <3 ppm, MS/MS ion scores >20, and expect values <1.

### Results

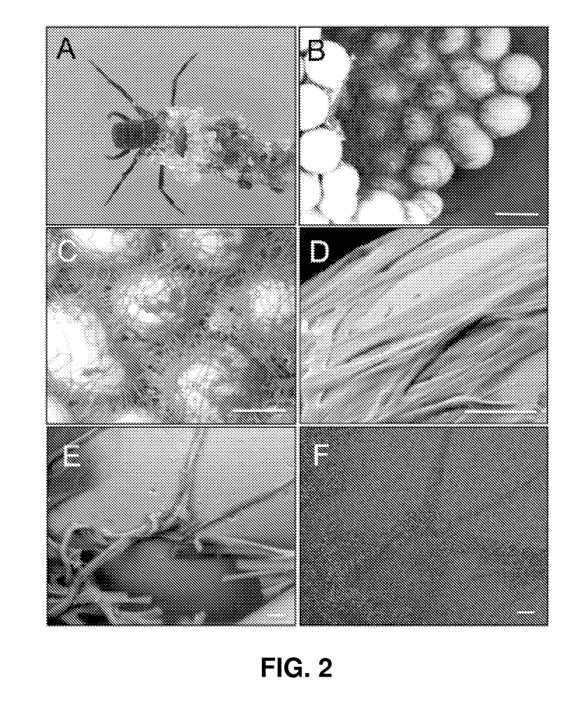

A local species (Brachycentrus echo) of stone case makers, known in the western mountain states as “Rock Rollers”, were collected from the lower Provo River in Utah to further investigate the molecular adaptations of underwater silk. When the stone cases were partially or completely taken away and the larva supplied with glass beads they rebuilt glass cases (FIG. 2A). Examination of the glass cases by scanning electron microscopy (SEM) revealed the beads had been ‘stitched’ together on the inside of the tube with silk fibers (FIGS. 2B and C), which appeared to be paired, flattened ribbons with a clear seam between the ribbons (FIGS. 2C and D). High resolution SEM images revealed a fibrous substructure in the fibers (FIG. 2D). Concentrated phosphorus was found to be coincident with the silk fibers by energy dispersive x-ray spectroscopy (EDS) (FIGS. 2E and F). Phosphorus was not detected in the silkworm silk.

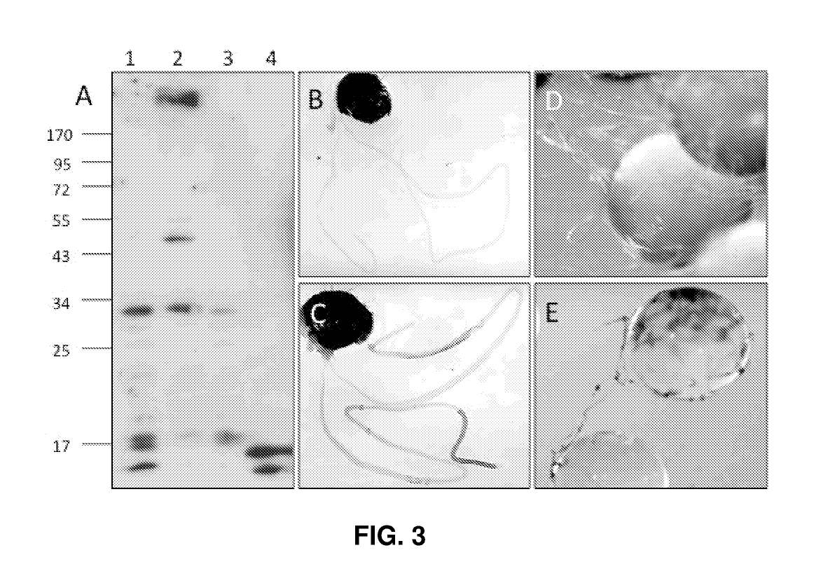

The presence of phosphorus in the form of phosphorylated serine (pS) was confirmed with an antibody against phosphoserine (α-pS) on western blots with protein isolated from the caddisfly silk glands. Phosphorylated bands were detected at MW >200 kDa (consistent with H-fibroin), at ˜50 kDa, at ˜30 kDa (the approximate MW expected for L-fibroin), and at 17 kDa and below (FIG. 3A, lanes 1,2). The band pattern depended on the extraction method; sodium dodecyl sulfate (SDS) solubilized caddisfly H-fibroin while 8M urea did not. Proteins extracted from the silk gland of B. mori with either urea or SDS and probed with α-pS did not have bands corresponding to H-fibroin but did have weak immunoreactive bands at ˜30 kDa and below 17 kDa (FIG. 3A, lanes 3,4). Further confirmation of phosphorylated silk proteins was obtained by immunostaining isolated caddisfly larval silk glands with anti-pS. The posterior region of the paired glands stained for pS (FIG. 3C). Silk fibers on glass beads retrieved from cases were also strongly labelled with the anti-pS antibody (FIG. 3E).

Caddisfly silk proteins isolated from dissected silk glands were heat denatured, rapidly cooled, and digested with trypsin. Tryptic peptides enriched for phosphopeptides were isolated by immobilized metal affinity chromatography (IMAC) and analyzed by tandem mass spectrometry. Experimental peptide masses were compared using the Mascot search engine against peptide masses calculated from translated caddisfly fibroin sequences deposited in GenBank. Genbank contains partial H-fibroin sequences for H. augustipennis, L. decipiens, and R. obliterata and complete L-fibroin sequences for all three caddisfly species. Eighteen unique peptides were identified, 16 of which were in most cases multiply phosphorylated (Table 1). The central regions of caddisfly H-fibroins are repeating sets of unique repeats that have been assigned letters A-F. All four species share a conserved D repeat that is shown in Table 1. Together the identified peptides spanned an entire D repeat taken at random from the L. dicipiens H-fibroin sequence. Identification of peptides in B. echo silk with the same sequence as L. decipiens H-fibroin demonstrates these species are closely related. Conservation of the position in all four species of two (SX)₄ motifs suggests the B. echo phosphorylation pattern is likely conserved as well. The larger repeating motif of two phosphorylated blocks flanking a hydrophobic region with a central proline must be an important structural element of caddisfly silks. The L. decipiens F repeats contain (SX)₃₋₅ motifs in 15-18 residue tryptic peptides but corresponding peptides or phosphopeptides were not identified in the B. echo mass analysis. The peptides may not be exactly conserved in B. echo, or the site(s) may not be accessible to trypsin. Likewise, no phosphoproteins from L-fibroin were identified.

Beads recovered from glass cases constructed by E. echo in a laboratory aquarium were lyophilized and subjected to amino acid analysis after hydrolysis in 50% HCl. The amino acid composition of the acid digested silk fibers was comparable to amino acid compositions of the other three caddisfly species deduced from the partial H-fibroin sequences in GenBank. The alanine content was higher but this is likely due to the comparison of whole silk fibers to H-fibroin only. The L. decipiens L-fibroin, for example, contains 14 mol % alanine. A similar mol% alanine in the B. echo L-fibroin and a 1:1 ratio of H- to L-fibroin would bring the composition in line with the other caddisflies. To estimate the ratio of phosphate to serine residues, aliquots from two of the acid hydrolysates were also analyzed by inductively coupled plasma-optical emission spectroscopy. There was no appreciable serine or P in background measurements made with unglued beads collected from the same area of the aquarium at the same time as the glass cases. The caddisfly silk contained 114 nmol of P corresponding to 166 nmol of serine in the hydrolysate and 164 nmol P to 256 nmol serine in the second hydrolysate for ratios of 0.69 and 0.64, respectively. These estimates seem reasonable given the ratio of phosphorylated serines found by mass spectrometry. The second most abundant element in the silk proteins was Ca²⁺ at ratios to P of 0.5 and 0.7 (Table 3).

### Discussion

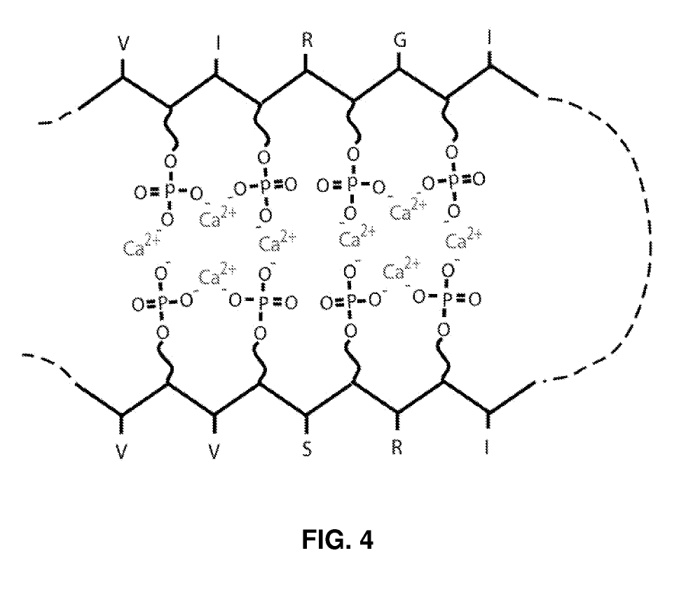

The B. echo silk proteins contain a two- to three-fold excess of negative relative to positive charges (assuming 60% of the serines are phosphorylated) that must be balanced by small counter ions (Tables 2 and 3). Association of the observed silk fiber Ca²⁺ with the phosphate side chains could create intra- and/or intermolecular cross-bridging of the (pSX)ₙ motifs into rigid domains analogous to the β-crystalline regions of spider and silkworm silks (FIG. 4). Indeed, x-ray diffraction studies of several caddisfly silks provided evidence of a repeating three-sheet ordered structure despite the absence of alanine. Formation of Ca²⁺ crossbridged phosphoserine domains would also contribute to dehydration of the predominantly hydrophilic silk proteins while submerged in water because the solubility of polyphosphates and Ca²⁺ is low at neutral pH. This role would be analogous to water exclusion by extensive β-sheet formation in dry silks.

At a longer length scale, phase separation of alternating hydrophilic and hydrophobic blocks is a major aspect of silk fiber assembly models for both spiders and silkworms. Their amphiphilic structures may lead first to liquid crystal or micelle formation in the posterior silk gland, then fibril formation as staggered amphiphilic blocks associate laterally during stress-induced elongation of silk proteins during fiber extrusion. Aquatic caddisflies may use a mechanism with broad similarities but key variations. Rather than alternating hydrophilic and hydrophobic blocks, staggered electrostatic association of alternating blocks with opposite charge may drive liquid-liquid phase separation as complex coacervates. In subsequent steps in the fiber formation process, stress-induced elongation and reorganization of the coacervated protein phase could lead to nanofibril formation, additional charge neutralization and dehydration of the fiber during extrusion. Perfect registry of the oppositely charged segments could cause the proteins to precipitate, while some imperfections in charge alignments would result in retained counter ions and water to provide localized plasticity.

The caddisfly H-fibroins share several structural design features with moth H-fibroins: non-repetitive N- and C-termini flanking a long central region of conserved motifs arranged in repeating blocks, regularly alternating hydrophobic and hydrophilic regions in the central core, and conserved positions and spacing of cysteine residues that covalently crosslink H- and L-fibroins. At the amino acid level the commonalities include a preponderance of simple motifs like GX, GGX, GPGXX, and SXSXSX, which is reflected in the high levels of G and S in both caddisfly and moth H-fibroins (Table 2). A conspicuous difference in amino acid composition is the comparatively low incidence of alanine in caddisfly, which in moth and spider H-fibroins occurs in runs of poly(A) and poly(GA) that confer β-crystallinity and mechanical strength to their silk fibers. Another striking difference is the high concentration (around 15 mol %) of positively charged basic residues, especially arginine, which are comparatively scarce in moth silks. Neither a cDNA nor protein homolog of P25 could be identified in any of the three caddisfly species. The important role of P25 in moth silk filament assembly and secretion suggests this may be another important distinction in the processing and assembly of dry versus wet silks.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

### Tables

TABLE 1
Phosphorylated peptides identified by tandem mass spectrometry.

| | | | |
|---|---|---|---|
| | | E | MS/MS |
| Peptide Sequences | Mᵣ₍ₑₓₚₜ₎ | (ppm)* | Score |

| | | |
|---|---|---|
| Ha | 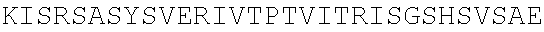 | (SEQ ID NO 1) |
| Sm | 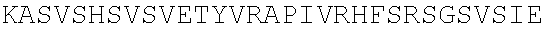 | (SEQ ID NO 2) |
| Ro | 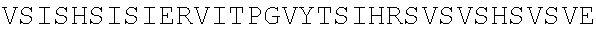 | (SEQ ID NO 3) |
| Ld | GKKVSISRSGSIERIVTPGYVTKISRSSSVSVEGGRRRGPWGYGR | (SEQ ID NO 4) |

| | | | | |
|---|---|---|---|---|
| 1 | GKVSISR (SEQ ID NO 5) | 825.4113 | 0.6 | 36 |
| 2 | SVSIER (SEQ ID NO 6) | 769.3379 | 1.1 | 31 |
| 3 | SVSIER (SEQ ID NO 7) | 849.3039 | 0.5 | 21 |
| 4 | RGKVSISRSGSIER (SEQ ID NO 8) | 1892.7713 | 0.01 | 21 |
| 5 | GKVSISRSGSIER (SEQ ID NO 9) | 1656.6968 | −3.3 | 29 |
| 6 | GKVSISRSGSIER (SEQ ID NO 10) | 1736.6708 | 0.4 | 24 |
| 7 | IVTPGVYTK (SEQ ID NO 11) | 976.5596 | 0.2 | 47 |
| 8 | IVTPGVYTKISR (SEQ ID NO 12) | 1412.7430 | 0.1 | 68 |
| 9 | TPGVYTK (SEQ ID NO 13) | 764.4076 | 1.0 | 41 |
| 10 | VTPGVYTKISR (SEQ ID NO 14) | 863.4756 | 0.4 | 43 |
| 11 | TPGVYTKISR (SEQ ID NO 15) | 1200.5903 | 0.1 | 56 |
| 12 | TPGVYTKISR (SEQ ID NO 16) | 1200.5903 | −0.1 | 50 |
| 13 | PGVYTKISR (SEQ ID NO 17) | 1099.5428 | 0.1 | 48 |
| 14 | PGVYTKISR (SEQ ID NO 18) | 1099.5413 | −1.3 | 40 |
| 15 | ISRSSSVSVEGGR (SEQ ID NO 19) | 1639.5453 | 0.4 | 29 |
| 16 | ISRSSSVSVEGGR (SEQ ID NO 20) | 1639.5453 | 0.4 | 29 |
| 17 | SSSVSVEGGR (SEQ ID NO 21) | 1123.3953 | 0.4 | 35 |
| 18 | SSSVSVEGGR (SEQ ID NO 22) | 1203.3613 | 0.1 | 34 |

The top four peptide rows are the conserved D repeats from H. augustipennis (Ha), S. marmorata
(Sm), R. obliterata (Ro), and L. dicipiens (Ld). Phosphorylated residues are bold and underlined.
Conserved serines are shaded.
*Experimental relative molecular mass (Mᵣ₍ₑₓₚₜ₎) error E = (Mₑₓₚₜ-M_{calc})/M_{calc} are presented in parts
per million (ppm). The MS/MS ion score is −10(LogP) where P is the probability the observed
peptide is a random match.

TABLE 2
Amino acid composition of four caddisfly and two moth species.

| | B. echo^{a} | L. decipiens | R. obliterata | H. augustipennis | B. mori | G. mellonella |
|---|---|---|---|---|---|---|
| Residue | (mol % ± s.d.) | (mol %) | (mol %) | (mol %) | (mol %) | (mol %) |
| Gly | 20.1 ± 0.4 | 24.6 | 24.9 | 19.4 | 45.9 | 28.6 |
| Ala | 6.3 ± 1.1 | 0.4 | 1.9 | 4.5 | 30.3 | 21.3 |
| Ser | 15.4 ± 1.8 | 17.2 | 14.7 | 12.5 | 12.1 | 17.0 |
| Thr | 3.2 ± 0.5 | 2.3 | 1.9 | 2.5 | 0.9 | 3.2 |
| Ile | 3.8 ± 0.4 | 4.3 | 9.1 | 6.6 | 0.2 | 4.2 |
| Leu | 6.0 ± 0.6 | 5.0 | 9.5 | 5.4 | 0.1 | 6.6 |
| Val | 4.1 ± 0.5 | 12.2 | 5.9 | 9.4 | 1.8 | 6.2 |
| Tyr | 4.1 ± 0.8 | 2.7 | 1.8 | 6.2 | 5.3 | 0.5 |
| Phe | 1.2 ± 0.06 | 0.8 | 0.2 | 0.1 | 0.6 | 0.4 |
| Pro | 4.0 ± 0.3 | 4.8 | 3.3 | 9.6 | 0.3 | 3.8 |
| Asx | 11.7 ± 1.6 | 2.6 | 3.8 | 3.4 | 0.9 | 2.8 |
| Glx | 3.5 ± 0.4 | 3.5 | 4.0 | 3.8 | 0.8 | 2.5 |
| Arg | 8.8 ± 0.5 | 14.1 | 7.7 | 9.6 | 0.3 | 1.6 |
| His | 0.7 ± 0.2 | 0.2 | 6.2 | 2.7 | 0.1 | 0.1 |
| Lys | 4.2 ± 0.4 | 2.3 | 3.3 | 2.1 | 0.2 | 0.3 |

^{a}Experimental amino acid composition from four independent analyses of B. echo silk. The amino acid compositions of the other species were deduced from H-fibroin sequences available in GenBank.

TABLE 3
Elements in B. echo silk protein.

| | Sample1 | Sample2 |
|---|---|---|
| Element | (nmol ± sd) | (nmol ± sd) |
| Ca | 55.7 ± 3.39 | 123.0 ±3.16 |
| Fe | 14.4 ± 0.07 | 28.7 ± 0.06 |
| Mg | 24.0 ± 0.09 | 15.4 ± 0.11 |
| Mn | 1.0 ± 0.0 | 0.90 ± 0.0 |
| Zn | 3.1 ± 0.02 | 3.5 ± 0.01 |
| S | 38.5 ± 0.35 | 55.4 ± 0.23 |
| P | 114.2 ± 0.48 | 164.2 ± 0.14 |

The amounts are nmols per 70 mg of glass beads from caddisfly cases after background subtraction. Backgrounds were determined with an equivalent mass of non-bonded glass beads collected from the same aquarium.

1. An adhesive complex coacervate comprising electrostatically associated block copolymers, wherein the block copolymers comprise alternating polycationic blocks and polyanionic blocks.

2. The coacervate of claim 1, wherein the polycationic block comprises a biodegradable polyamine.

3. The coacervate of claim 2, wherein the biodegradable polyamine comprises a polysaccharide, a protein, a synthetic polyamine, or any combination thereof.

4. The coacervate of claim 2, wherein the biodegradable polyamine comprises an amine-modified natural polymer.

5. The coacervate of claim 1, wherein the polycationic block comprises at least one fragment comprising the formula I 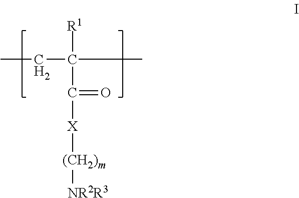
wherein R¹, R², and R³ are, independently, hydrogen, an alkyl group, or a guanidinium group, X is oxygen or NR⁵, where R⁵ is hydrogen or an alkyl group, and m is from 1 to 10, or the pharmaceutically-acceptable salt thereof, wherein at least one of R² or R³ is an actinically crosslinkable group.

6. The coacervate of claim 1, wherein the polyanionic block comprises a polyphosphate compound.

7. The coacervate of claim 1, wherein the polyanionic block comprises a polyacrylate comprising one or more pendant phosphate groups.

8. The coacervate of claim 1, wherein the polyanionic block comprises a polymer comprising at least one fragment comprising the formula II 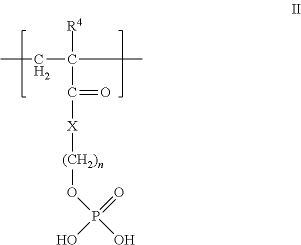
wherein R⁴ is hydrogen or an alkyl group, X is oxygen or NR⁵, where R⁵ is hydrogen or an alkyl group, and n is from 1 to 10, or the pharmaceutically-acceptable salt thereof.

9. The coacervate of claim 1, wherein the coacervate further comprises at least one multivalent cation, and the multivalent cation comprises Ca⁺² and/or Mg⁺².

10. The coacervate of claim 1, wherein the coacervate further comprises one or more bioactive agents encapsulated in the coacervate.

11. The coacervate of claim 10, wherein the bioactive agent comprises an astringent.

12. The coacervate of claim 1, wherein the astringent comprises an inorganic salt of aluminum, iron, zinc, manganese, bismuth, or any combination thereof.

13. The coacervate of claim 11, wherein the astringent comprises ferric sulphate, ferric subsulphate, ferric chloride, zinc chloride, aluminum chloride, aluminum sulfate, aluminum chlorohydrate, aluminum acetate, aluminum potassium sulfate, aluminum ammonium sulfate, or any combination thereof.

14. The coacervate of claim 1, wherein the polyanionic block comprises at least one dihydroxyl aromatic group capable of undergoing oxidation, wherein the dihydroxyl aromatic group is covalently attached to the polyanion.

15. The coacervate of claim 1, wherein the coacervate further comprises a stabilized oxidant complex.

16. The coacervate of claim 1, wherein the block copolymers are crosslinked with one another via a Diels-Alder reaction,

17. The use of the coacervate of claim 1 to adhere a material or object to a wet substrate,

18. The use of claim 17, wherein the wet substrate comprises a metal substrate or glass.

19. A method for adhering a substrate to a bone of a subject comprising contacting the bone with the coacervate of claim 1 and applying the substrate to the coated bone.

20. The method of claim 19, wherein the substrate is a metal substrate, a backing material, a plastic film, or foil.

21. A method for adhering a bone-tissue scaffold to a bone of a subject comprising contacting the bone and tissue with the coacervate of claim 1 and applying the bone-tissue scaffold to the bone and tissue,

22. A method for delivering one or more bioactive agents comprising administering the coacervate of claim 1 to a subject.

23. A method for repairing a corneal laceration in a subject, comprising applying to the laceration the coacervate of claim 1.

24. A method for inhibiting blood flow in a blood vessel of a subject comprising introducing the coacervate of claim 1 into the vessel.

25. A method for inhibiting blood flow in a blood vessel of a subject comprising introducing electrostatically associated block copolymers into the blood vessel, wherein the block copolymers comprise alternating polycationic blocks and polyanionic blocks.

26. A water-based composition comprising the coacervate of claim 1.

27. The composition of claim 26, wherein the composition comprises a water-based paint,

28. The use of the coacervate of claim 1 as a pressure sensitive adhesive.

29. The use of claim 28, wherein the pressure sensitive adhesive is a medical adhesive.

30. A synthetic fiber produced by the coacervate of claim 1.

31. A fabric produced from the fibers of claim 30.
